(19) United States
(12) Patent Application Publication
(10) Pub. No.: US 2026/0192541 A1
(43) Pub. Date: Jul. 9, 2026

(54) COMPOSITE SHELL

(71) Applicant: Siemens Healthineers AG, Forchheim (DE)

(72) Inventors: Christian SEIDEL, Baiersdorf (DE); Felix NTOURMAS, Coburg (DE); Martin SEIFERT, Bayreuth (DE)

(73) Assignee: Siemens Healthineers AG, Forchheim (DE)

(21) Appl. No.: 19/443,282

(22) Filed: Jan. 8, 2026

(30) Foreign Application Priority Data
Jan. 9, 2025 (EP) 25150947.7

(51) Int. Cl. B32B 5/02; B32B 1/00; B32B 5/12; B32B 5/26

(52) U.S. Cl. CPC B32B 5/022; B32B 1/00; B32B 5/12; B32B 5/265; B32B 2250/05; B32B 2307/514; B32B 2307/7376; B32B 2479/00; B32B 2535/00

(57) ABSTRACT

One or more example embodiments relates to a composite shell such as for in large-scale medical devices and/or as part of a composite component resistant to flexural stress such as finds application for seats, couches, storage facilities and/or interior fittings of vehicles. Sustainability in the manufacture of large-scale medical devices comprising composite shells for the couchtop boards of patient couches is technically realized.

### CROSS-REFERENCE TO RELATED APPLICATION(S)

The present application claims priority under 35 U.S.C. § 119 to European Patent Application No. 25150947.7, filed Jan. 9, 2025, the entire contents of which is incorporated herein by reference.

### TECHNICAL FIELD

One or more example embodiments relates to a composite shell, for example, part of a patient couch and/or part of a composite component resistant to flexural stress such as for seats, couches, storage facilities and/or interior fittings of vehicles.

### RELATED ART

Patient couches according to the prior art have composite shells typically composed of fiber plastic composites and/or composite materials, for example with a hard foam core. A fiber-reinforced composite is generally a multiphase or mixed material comprising two main components—an embedding matrix and reinforcing fibers. In this arrangement, the reinforcing fibers and the embedding matrix take on very specific tasks. Reciprocal interactions between the two components endow the fiber-reinforced composite with superior properties to each of the two components involved individually. However, this also requires the two materials involved to possess very different properties.

Continuous fiber-reinforced plastics on the basis of

carbon fibers (CFRP) are utilized, for example. For example, duromers and/or thermoplastics, as well as arbitrary mixes and blends thereof, form an embedding matrix. The composite shells for patient couches, for example, comprise composite materials which in cross-section reveal hybrid structures composed of CFRP layers having different fiber orientations, the main flexural load in particular being oriented longitudinally along the component. In this case, for example, a layer structure of a composite shell is realized and 100 or more individual layers having different, to some extent opposite, fiber orientations are present.

Composite shells may be fabricated in what is termed a sandwich structure, wherein two flexurally resistant composite layer stacks enclose a core composed of a hard foam material and/or other core materials. These sandwich solutions can typically be produced by means of a press forming process consisting of wet layup laminating via hydraulic presses and/or autoclave with vacuum bag structure. A known sandwich structure of a patient couch is described for example in DE 102022205106.

There is also the monolithic structure, wherein the components or patient couches consist only of fiber laminate, including in cross-section. These are produced for example from the dry reinforcement fibers by means of liquid resin injection methods.

### SUMMARY

There is no prior art approach for incorporating the material of a composite shell, regardless of which construction, whether a monolithic or sandwich structure, in patient couches into a material cycle. There is therefore a need for technical solutions in which the material technology of composite shells, in particular of patient couches, is fed, at least to some extent, by a material cycle.

One or more example embodiments provides a sustainable material technology on the basis of recycled reinforcement fibers and/or on the basis of scrap fibers for a composite shell, for example, as part of a patient couch, with comparable technical properties to the composite shells exclusively composed of new fibers.

Accordingly, one or more example embodiments provides a composite shell in a sandwich structure, comprising two outer composite layer packs as external layers and centrally a core, wherein there is contained in the core and/or in the two surrounding outer composite layer packs nonwoven fabric-reinforced composite material which comprises fibers from recycling and/or production wastage rejects in the nonwoven fabric.

### BRIEF DESCRIPTION OF THE DRAWINGS

FIG. 1 shows a design example of a composite

shell according to an exemplary embodiment of the invention,

FIG. 2 shows an exemplary layer structure of a composite shell according to one or more example embodiments,

FIG. 3 shows the effect of the recycling modification on the flexural rigidity of a composite shell for an exemplary embodiment of the invention, and

FIG. 4 illustrates a radiotherapy device with a patient couch according to one or more example embodiments.

### DETAILED DESCRIPTION

Patient couches for receiving and/or positioning a patient and the latter's movement through the scan plane are used in large-scale medical devices such as computed tomography (CT) scanners, in molecular imaging (MI), in X-ray machines and in magnetic resonance tomography (MRT), as well as in the angiography environment (AT). In addition, patient couches are also employed in radiotherapeutic irradiation by means of X-rays or particle radiation, for corresponding radiation treatment planning or for medical intervention aided by imaging.

The construction of patient couches for medical applications always takes into account a mechanical stability and/or rigidity of the composite shell on the one hand and the permeability and/or the imaging and/or X-ray absorption behavior of the composite shell on the other, which behavior can have an impact on the image quality as well as the X-ray dose applied to the patient. Composite shells for patient couches are therefore preferably designed to be X-ray-transparent.

In this regard, there is on the one hand the requirement for the patient couch to be as lightweight and thin-walled as possible in order to keep the exposure to radiation and/or the dose rate for the patient to a minimum. On the other hand it is necessary to comply with mechanical specifications in relation to strength and stiffness since, for example, in real-world use heavy patients—weighing more than 125 kg for example—with high projections of the couchtop board—such as, for example, with 1.5 m lever arm—are also moved through the scan plane of the device. In such cases the composite shell of the patient couch must not only not break but must also not exceed a certain deflection tolerance measure in the region of the scan plane in order to keep the patient in the isocenter. Excessively large deflections potentially lead to negative results in terms of image quality and consequently in terms of the diagnosis.

A high mechanical stability is typically achieved by an increase in the material used, which—for example—lowers the X-ray transparency. The described design criteria of flexural strength on the one hand and lightness or transparency to the applied radiation on the other hand are therefore normally conflicting requirements.

According to an embodiment, the composite shell is part of a couchtop board of a patient couch.

According to a further embodiment, the core comprises nonwoven fabric-reinforced composite material.

According to an embodiment, the core of the composite shell comprises a composite material containing nonwoven fabric reinforcement in the form of bulk material.

According to a further embodiment, a composite layer pack comprises up to 300, in particular up to 150, individual layers.

According to an embodiment, the composite shell is assembled symmetrically around the core such that in terms of the number of individual layers, the two outer layers in the form of composite layer stacks have the same number of individual layers.

According to an embodiment, the composite shell is assembled symmetrically around the core such that the two composite layer stacks have an identical and/or mirrored, i.e. axially symmetrical, individual layer arrangement in terms of the orientation of the individual layers with continuous fiber reinforcement.

According to an embodiment, at least one individual layer of a composite layer pack comprises a fiber reinforcement with oriented continuous fibers.

According to an embodiment, at least one individual layer of a composite layer pack comprises nonwoven fabric reinforcement.

According to an embodiment, in at least one composite layer stack there is present a layer structure in which the fiber orientations of two successive individual layers having fiber reinforcement composed of oriented continuous fibers are rotated relative to one another through 90° in each case.

According to an embodiment, a sandwich structure having the following individual layer sequence is present in a composite shell:
2 or a multiple of 2 individual layers having fiber reinforcement and at least in part differently oriented continuous fibers,
core composed of bulk composite material with nonwoven fabric reinforcement,
2 or a multiple of 2 individual layers with fiber reinforcement and at least in part differently oriented continuous fibers.

According to a further embodiment, a sandwich structure having the following individual layer sequence is present in a composite shell:
2 or a multiple of 2 individual layers with fiber reinforcement, wherein at least two adjacent individual layers comprise oriented continuous fibers rotated through 90°,
in the core, multiple individual layers with nonwoven fabric reinforcement,
2 or a multiple of 2 individual layers with fiber reinforcement, wherein at least two adjacent individual layers comprise oriented continuous fibers rotated through 90°.

A sandwich structure, in the present context, denotes a structure which reveals a multilayer material solution, wherein two outer regions and a central region are present and the central region is also referred to as the core. In the borderline case, the well-known monolithic structure of a composite shell may also be included here under the term “sandwich structure” if the core and the surrounding composite layer stacks have the same construction, i.e. possess an identical multilayer material solution.

According to the prior art, the core in the conventional sandwich structure mainly comprises compact and/or foamed plastic solutions with and without fiber solutions.

In the present case, it is proposed in contrast thereto to make the core out of a—as bulk composite material core—and/or multiple individual layers with nonwoven fabric reinforcement, wherein the core, as already mentioned, is not necessarily different in respect of the composite material and layer stack structure from the surrounding outer layers.

The thickness or wall strength of the composite shell lies in the range of 0.5 to 100 mm, preferably 5 to 50 mm, and particularly preferably in the range of 7 to 35 mm.

In this case the thickness of the core lies in the range of 0.1 mm to 50 mm, preferably 0.5 mm to 30 mm and particularly preferably 0.7 mm to 20 mm.

Depending on the internal structure in the region of the thickness of the core, the thickness of the outer layers in the form of composite layer stacks is equal to or—for example—less than the thickness of the core; it accordingly lies between 0.1 to 30 mm, preferably in the range of 0.5 to 15 mm and particularly preferably in the range of 1 to 10 mm.

A typical thickness of an individual layer is approximately in the range of 0.1 to 1 mm.

The thickness of a nonwoven fabric-reinforced bulk composite material core as a molded body lies in the range of 0.1 to 50 mm.

A composite layer stack forms an outer layer and comprises a series of individual layers composed of composite material. The term “composite material” refers to a material composed of reinforcement fibers in a plastic matrix. The fiber reinforcement forms the “stiff” part of the fiber-reinforced plastic and is present for example in the form of fibers, fiber bundles, rovings, fiber non-crimp fabrics, traditional web structures and/or as nonwoven fabric.

The nonwoven fabrics comprise both nonwoven fabrics present in two-dimensional form, in the form of a lamination, layer, film and/or individual layer, for example, what are known as sheet materials, and also nonwoven fabrics that form three-dimensional and irregular fiber composites which, prior to the processing with resin to produce composite material, look like clouds and are what are termed bulk materials.

In the present context, the term “nonwoven fabric-reinforced”denotes a composite material comprising recycled and/or scrap fibers. The nonwoven fabric-reinforced composite material is part of a plastic cycle.

Reinforcement fibers can accordingly be present as continuous fibers, as fiber bundles, as individual fibers of a certain length, as fiber composites, as fiber non-crimp fabrics, as fiber woven fabrics and/or as nonwoven fabrics. In the cost-intensive primary textile manufacturing of expensive continuous fibers, such as carbon fibers, for example unidirectional (UD) carbon fibers and/or high modulus carbon fibers, as well as HT (high tenacity) carbon fibers, refuse known as “waste” materials accumulates which can be used in the form of nonwoven fabrics. In the present context these are also referred to as “scrap fibers”.

In contrast to the simple fiber, the woven fabric, the defined fiber composite and/or the defined fiber roving, a nonwoven fabric is an unordered statistical “nonwoven” arrangement of fibers and/or fiber fragments. Evidence of the use of nonwoven fabric fiber solutions in fiber-reinforced plastics is furnished for example by means of optical evaluation because a nonwoven fabric has different optic properties than a fiber woven fabric, fiber non-crimp fabric and/or fiber composite. Defined arrangements of fibers are generally characterized by traditional web structures, whereas a nonwoven fabric structure is arbitrary and unordered. For example, “nonwoven” is to be understood to refer to all types of the arrangement of fibers except for a regular structure such as is produced in a weaving process. Nonwoven fabrics are flexible two-dimensional textile sheet materials or three-dimensional bulk materials.

Typical fiber lengths in the nonwoven fabric lie in the range of a lower limit of 2 mm to 7 mm up to an upper limit of 300 mm to 400 mm. For example, they lie in the range of 8 mm to 170 mm, in particular in the range of 10 mm to 100 mm.

All common inorganic and/or organic reinforcement fibers, preferably recycled fibers and/or scrap fibers, as well as arbitrary combinations and blends, come into consideration as fibers suitable for the nonwoven fabric. This includes, for example, all types of carbon fibers, glass fibers, organic fibers, such as, for example, aramid fibers, PET (polyethylene terephthalate) fibers, PP (polypropylene) fibers, cellulose, ceramic fibers—for example metal oxides, such as, for example, corundum (Al2O3) fibers, and silicon carbide fibers. The aforementioned fibers can be used individually or in a mix, as well as in any combinations in the nonwoven fabric.

For example, different fiber types and/or fiber qualities can be used bundled in rovings, or as pultruded laminate, combined for assembling a nonwoven fabric. By means of the combinations of different reinforcement fibers, all kinds of physical and chemical properties of the resulting nonwoven fabric fiber-reinforced composite material can be selected and influenced in a targeted manner.

For example, a duromer resin, based on one of the compounds, for example, glycidyl ether, novolaks, epoxy resins, vinylester resins, polyurethanes, polyesters, silicones, polyethylene, ultrahigh molecular polyethylene, as well as any desired mixes, blends, copolymers of the aforementioned compounds, as described above, are suitable for use as a polymer matrix. Furthermore, different thermoplastics such as polyamide, polyethylene terephthalate, polypropylene, again as well as any desired mixes, blends, copolymers of the aforementioned compounds, can be used.

Nonwoven fabrics can produce two-dimensional flat composite materials or also bulk materials by layering of individual layers. For example, sheet molding compounds (SMCs) or bulk molding compounds (BMCs), also nonwoven fabric-reinforced, can also be produced using recycled fibers and/or, in the present case, used for constructing a composite shell.

Different fibers, fiber residues and recycling fibers are used to produce the nonwoven fabrics. Recycling fibers, i.e. fibers recovered from used materials and/or products, are known. These fibers are available partly as woven fabric, as knitted fabric, as interlooped and/or interlaced yarns. There are also scrap fibers which, though factory-fresh, come from process waste such as clippings, production wastage rejects and the like.

To produce the composite material, the fiber reinforcement is submitted for example and saturated or impregnated with liquid resin and/or immersed in a bath containing resin. Next, the compound formed as a result is hardened by means of temperature, drying, etc., which causes the fiber reinforced plastic to be formed into the composite material. Depending on the resin, the polymerization of the initially liquid matrix material is realized using hardener as an additive and/or radical polymerization or without hardener as a homopolymerization using a starter or initiator.

In order to form the composite layer stack, multiple thin individual layers from the composite material are placed one on top of the other and then the thus constructed layer pack is packed for example into a film bag, evacuated under pressure, placed into an autoclave and thermoformed at an autoclave pressure of approx. 10 bar and increased temperature.

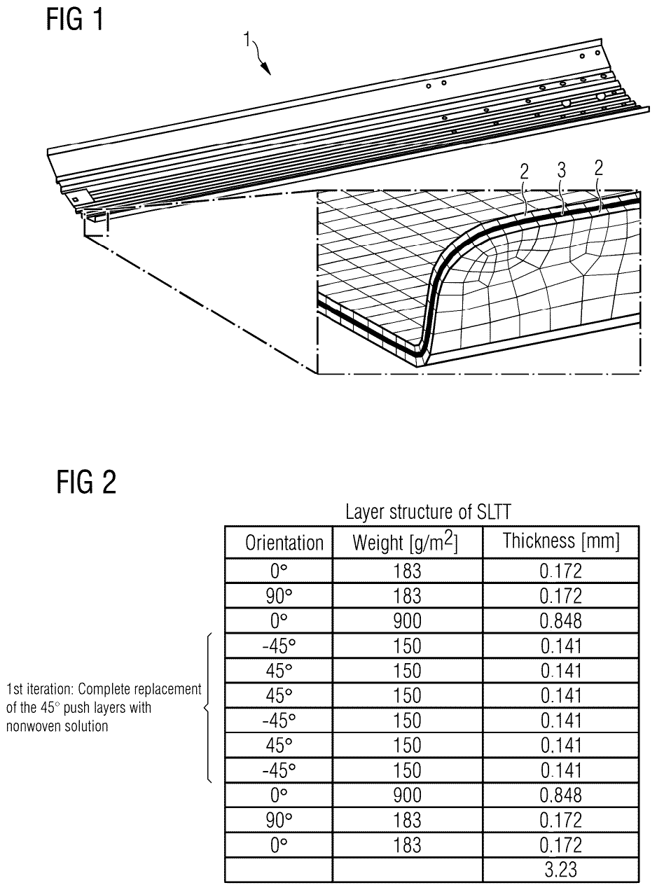

A composite shell 1 is illustrated in FIG. 1. The composite shell 1 features a shell shape, as is typical for the design of couchtop boards, for example for patient couches. FIG. 1 also shows a detail from the composite shell 1 in enlarged form, making the sandwich structure clearly recognizable. The two outer layers in the form of composite layer stacks 2 surrounding a core 3 can be seen. The composite layer stacks 2 each comprise a series of individual layers which are present in each case as either fiber-reinforced or nonwoven fabric-reinforced. The core 3 comprises for example nonwoven fabric-reinforced composite material either in the form of individual layers and/or in the form of a bulk material.

“Fiber reinforced” in the present context is the term denoting when a fiber reinforcement using fibers, for example continuous fibers, is present in the composite material, while “nonwoven fabric reinforced” analogously refers to when a nonwoven fabric—with an appropriate proportion of recycled fibers—is present as a rigid component in the composite material.

FIG. 2 shows a table detailing the layer structure of a composite shell 2. The table shows a fiber reinforcement with the orientation of the reinforcement fibers such that the orientation data relating to the individual layer [specification of the angle °] of the continuous fibers can be read off in the leftmost column, the data relating to the weight of the individual layer in [g/m2] in the middle column, and the data relating to the thickness of the layer in [mm] in the rightmost column. The orientations of the individual layers with fiber reinforcement are accordingly arranged at 0° in the longitudinal direction, at 90° in the transverse direction, and at +/−45° direction such that approximately the same properties are produced in all directions and therefore the composite shell withstands longitudinal, transverse and/or torsional stresses equally well.

In a test, six middle individual layers from the composite shell were now replaced by nonwoven fabric-reinforced individual layers comprising recycled fiber material and/or waste and/or scrap fiber material and the two composite shells were compared with one another in respect of their flexural rigidity.

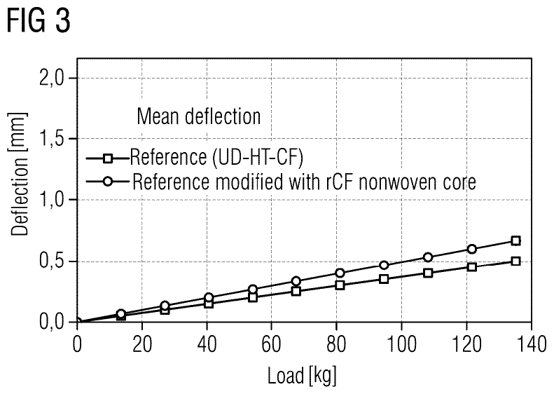

FIG. 3 shows the results of the tests as described above in the form of a graph. The load in [kg] has been plotted in the abscissa and the deflection in [mm] in the ordinate. The lower graph containing the square-shaped measurement points shows a conventionally constructed, as in the table from FIG. 2, composite shell with exclusively individual layers composed of oriented continuous individual fiber reinforcement and, fractionally above this, i.e. only marginally less bend-proof, a modified composite shell—according to an exemplary embodiment of the invention—comprising nonwoven fabric-reinforced individual layers, the measurement values of which are represented in the graph by circle-shaped measurement points.

It can clearly be seen that the component stiffness of a composite shell is only slightly inferior when the expensive continuous fibers in the fiber reinforcement are replaced by nonwoven fabric reinforcement composed of recycled and/or scrap fibers.

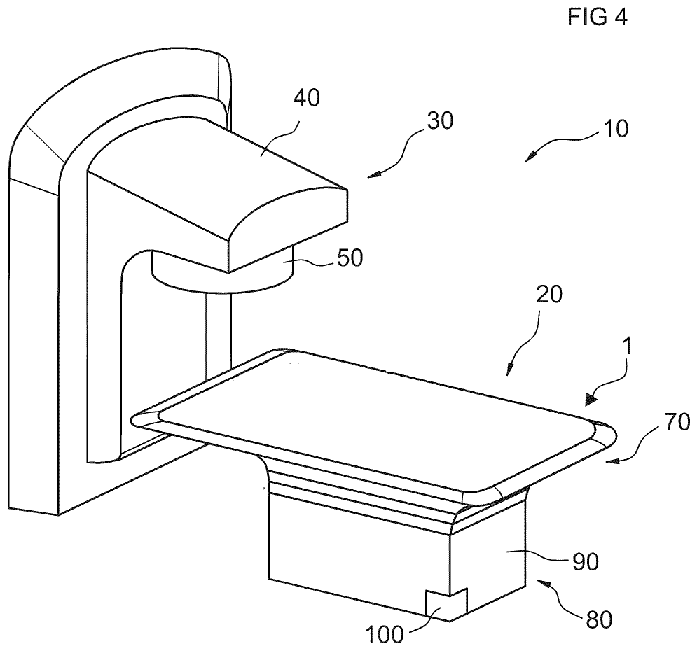

FIG. 4 shows a radiotherapy device 10. The radiotherapy device 10 comprises a patient couch 20 and a linear accelerator 30. The linear accelerator 30 includes a gantry 40 and a beam outlet 50. The linear accelerator 30 and the patient couch 20 are arranged such that a patient positioned on the patient couch 20 can be treated with radiation from the linear accelerator 30 during radiotherapy treatment.

The patient couch 20 comprises a couch board section 7 and a base section 80. The couch board section 70 includes the composite shell 1 in a sandwich structure.

The couch patient section 70, especially the upper part of the patient couch 20, provides accommodation and fixation for the patient. The couch board section 70 preferably includes a flat or curved surface and defines a horizontal plane. The surface of the couch board section 70 is preferably transparent to x-ray and high-energy photon beams.

The base section 80, particularly the lower part of the patient couch 20, connects the patient couch 20 to the ground and may include mechanisms for vertical and horizontal movement of the patient. The couch board section 80 include mechanisms for rotational and tilting movements, allowing optimal positioning of the patient for treatment. The base section 80 is preferably covered and/or shielded by a cover 90.

The radiotherapy device 10 and patient couch 20 include a control unit 100, configured to control and/or steer the rotational and tilting movements, as well as the horizontal and vertical movements of the patient couch 20. The control unit 100 may also be integrated with the control unit of the linear accelerator 30, ensuring coordinated positioning and treatment of the patient with radiation.

It could be demonstrated here that sustainability in the manufacture of composite shells is possible because the nonwoven fabric reinforcement of individual layers—depending on the installation of the individual layers into the composite layer stacks of the sandwich structure—yields almost equally good test results with regard to flexural rigidity as the pure fiber reinforcement. Accordingly, a usable recirculation of recycled fibers, as well as of fiber scraps, is technically achievable in the production of composite shells.

Advantages result in particular with regard to resource efficiency, in the use of recycling material, and in terms of the ecological and economic footprint. In this connection, the fiber recycling can be considered as having a material CO2 footprint of zero. The sustainability improvements for a high-energy raw material are supported and promoted in multiple aspects as a result.

In real-world use, the cited components are partly or completely replaced by materials composed of “recycled carbon fibers” rCF-based or nonwoven fabrics made of other materials, for example in the center of the cross-section in the vicinity of the neutral fibers under flexural load.

A further advantage for the cited replacement of +/−45° continuous fiber layers is a cost benefit in the case of the rCF materials as nonwoven fabrics since the expensive CF fiber production is dispensed with.

Sustainability in the manufacture of large-scale medical devices comprising composite shells for the couchtop boards of patient couches is technically realized for the first time. It is accordingly possible—by targeted use of nonwoven fabrics instead of the expensive continuous fibers in composite shells, for example for couchtop boards of patient couches, car seats and other composite components subject to bending loads—to build components that are mechanically of virtually the same quality using recycled reinforcement nonwoven fabrics composed of recycled and/or scrap fibers instead of using freshly produced continuous fibers.

Independent of the grammatical term usage, such as “patient”, for example, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure.

Specific structural and functional details disclosed herein are merely representative for purposes of describing example embodiments. The present invention may, however, be embodied in many alternate forms and should not be construed as limited to only the embodiments set forth herein.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A composite shell in a sandwich structure, the composite shell comprising:
two outer composite layer packs as outer layers; and
a central core, at least one of the central core or the two outer composite layer packs includes a nonwoven fabric-reinforced composite material including fibers from at least one of recycling or production wastage rejects.

2. The composite shell of claim 1, wherein the central core comprises recycled fibers in a nonwoven fabric reinforcement.

3. The composite shell of claim 1, wherein the composite shell is part of a patient couch for at least one of diagnostic or therapeutic medical equipment.

4. The composite shell of claim 1, wherein the central core of the composite shell comprises composite material containing nonwoven fabric reinforcement as a bulk material.

5. The composite shell of claim 1, wherein up to 300 individual layers are in at least one of the composite layer packs.

6. The composite shell of claim 1, wherein the composite shell is constructed symmetrically around the central core.

7. The composite shell of claim 1, wherein the composite shell comprises at least one individual layer in at least one of the composite layer packs, the at least one individual layer comprising a fiber reinforcement of oriented continuous fibers.

8. The composite shell of claim 1, which comprises at least one individual layer in a composite layer pack having a fiber reinforcement composed of nonwoven fabric reinforcement.

9. The composite shell of claim 1, wherein at least one composite layer stack includes a layer structure in which fiber orientations of two successive individual layers having fiber reinforcement composed of oriented continuous fibers are rotated relative to one another through 90°.

10. The composite shell of claim 1, a wall thickness of the composite shell is in a range of 0.1 to 100 mm.

11. The composite shell of claim 1, wherein
a first outer layer includes two or a multiple of two individual layers having fiber reinforcement and at least in part differently oriented continuous fibers,
the central core is composed of bulk composite material with nonwoven fabric reinforcement, and
a second outer layer includes two or a multiple of two individual layers with fiber reinforcement and at least in part differently oriented continuous fibers, and the first outer layer, the central core, and the second outer layer are layered sequentially.

12. The composite shell of claim 1, wherein
a first outer layer includes two or a multiple of two individual layers with fiber reinforcement, wherein at least two adjacent individual layers of the first outer layer have oriented continuous fibers rotated through 90°,
the central core includes multiple individual layers with nonwoven fabric reinforcement, and
a second outer layer includes two or a multiple of two individual layers with fiber reinforcement, wherein at least two adjacent individual layers of the second outer layer have oriented continuous fibers rotated through 90°, and the first outer layer, the central core, and the second outer layer are layered sequentially.

13. The composite shell of claim 1, comprising at least one of a sheet molding compound material (SMC) or a bulk molding compound material (BMC).

14. The composite shell of claim 2, wherein the central core of the composite shell comprises composite material containing nonwoven fabric reinforcement as a bulk material.

15. The composite shell of claim 14, wherein up to 300 individual layers are in at least one of the composite layer packs.

16. The composite shell of claim 15, wherein the composite shell is constructed symmetrically around the central core.

17. The composite shell of claim 16, wherein the composite shell comprises at least one individual layer in at least one of the composite layer packs, the at least one individual layer comprising a fiber reinforcement of oriented continuous fibers.

18. The composite shell of claim 2, which comprises at least one individual layer in one of the composite layer packs having a fiber reinforcement composed of nonwoven fabric reinforcement.

19. The composite shell of claim 2, wherein at least one composite layer stack includes a layer structure in which fiber orientations of two successive individual layers having fiber reinforcement composed of oriented continuous fibers are rotated relative to one another through 90°.

20. A patient couch comprising:
composite shell in a sandwich structure, the composite shell including,
two outer composite layer packs as outer layers, and
a central core, at least one of the central core or the two outer composite layer packs includes a nonwoven fabric-reinforced composite material including fibers from at least one of recycling or production wastage rejects.
